(19) United States
(12) Patent Application Publication
(10) Pub. No.: US 2008/0213129 A1
(43) Pub. Date: Sep. 4, 2008

(54) NON-LINEAR UV LIGHT SOURCES FOR DISINFECTION

(75) Inventors: Adrianus Johannes Hendricus Petrus van der POL, Heeswijk-Dinther (NL); Jaak GEBOERS, Neerpelt (BE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/019,890

(22) Filed: Jan. 25, 2008

Related U.S. Application Data
(60) Provisional application No. 60/886,519, filed on Jan. 25, 2007.

(51) Int. Cl. A61L 2/00

(52) U.S. Cl. 422/24; 422/121

(57) ABSTRACT

The present disclosure provides for systems and methods for disinfecting of components of an HVAC system. At least one non-linear UV light source is positioned within the HVAC system. A non-linear UV light source can include but is not limited to a spherical bulb lamp or a circular lamp. Non-linear UV light sources are positioned about desired HVAC components to deliver UV light to the surface of those components thereby preventing unwanted biological growth. A plurality of non-linear light sources can be positioned throughout the HVAC system including about a heating or cooling coil apparatus, an air moving apparatus such as a fan positioned within an air duct and/or within the air duct to disinfect air flow passing through the air duct.

The present disclosure relates to systems and methods for disinfecting and sanitizing cooling coils of HVAC systems using non-linear UV light sources.

A particular industry that is economically sensitive to costs is the heating, ventilation and air conditioning (HVAC) industry. HVAC systems are typically comprised of fans and ductwork for moving air where needed. An HVAC system will include a cooling and heating section for, respectively, cooling and heating the air. In most HVAC systems, air is drawn in, filtered, cooled and dehumidified or heated and humidified, and then delivered to a room. The greatest portion of this air is drawn from the conditioned space for recirculation through the HVAC system. Considerable effort has been made to make these components more efficient.

One of several methods of saving energy in an HVAC system includes the use of variable frequency drives on any motor used in a HVAC system. When and if the system load decreases, this can be sensed and the motors in the HVAC system will be slowed to an equilibrium value to save motor energy consumption. Another method is to reduce the amount of outdoor air to eliminate having to condition it. Another method is an economizer cycle that utilizes 100% outdoor air when ambient temperature is suitable for cooling the space. Another method is to replace aging equipment with newer and more efficient equipment.

Another factor impacting design and operation of HVAC systems is indoor air quality (IAQ). One major factor in IAQ is the amount of outdoor air introduced into an otherwise sealed space serviced by an HVAC system. The HVAC industry has adapted standards for the introduction of outdoor air into spaces serviced by an otherwise closed HVAC system. These include offices, residential, commercial, industrial and institutional spaces, as well as the interior of vehicles such as cars, buses, planes and ships. In addition to controlling indoor air for occupant comfort, the goal of HVAC systems is to provide air with reduced levels of particulates, gases and bioaerosols, be it for semiconductor, pharmaceutical or food processing facilities, hospitals, schools or offices and the home.

Many ventilation systems include a cooling section. The cooling section includes a type of heat exchanger typically referred to as a “cooling coil,” through which air is forced and cooled. Cooling coils typically are made using aluminum fins over refrigerant tubes which have been formed into a desired shape. Essentially the same coil arrangement is used in most cooling systems, whether in HVAC systems are designed for occupied spaces, or for refrigerators and freezers.

A similar configuration is often used in heating sections, though the thermodynamic operation is opposite to that in a cooling section. The heat exchanger of a heating section often comprises a coil, and water of an elevated temperature passing through the coil. The heating coil is fashioned in a manner to promote heat transfer from the water to the heating coil. The heating coil is further fashioned to promote heat transfer from the heating coil to air which is forced across and through the heating coil.

As a normal consequence of the process of cooling air, several things occur. One is that humidity (latent heat) is removed from the air. This moisture collects on the coil fins and/or anything else nearby which is below dew point, including the ductwork. Typically, a drain pan is positioned below a cooling coil. The collected moisture runs down the fins and into the drain pan under the force of gravity. Water, which collects in the drain pan, typically flows away through a drain pipe equipped with a trap.

A further consequence is organic matter depositing and/or collecting on the cooling coil fins from the air passing over them. Though the fins of the cooling coil appear to be smooth, in many cases, when viewed under a microscope, they can be seen to have an irregular and somewhat pitted surface. The organic matter can therefore adhere easily to the damp and rough surface of the cooling coil.

Cooling sections of a given duct system are often dark and at off times, can be warm. Though when operating it will be quite cold, the cooling section will have varying cycles of cooling. When not cooling, the cooling coils typically reach room temperature. Similar effects are encountered with heating coils, though typically to a lesser degree than with cooling coils. Altogether, these consequences can yield an environment in which molds and bacteria can grow and thrive. Over time, a heat exchanger can become near fully encrusted with microorganism activity bound to an organic substrate. The drain and drain pans also become a growth environment for mold and bacteria. The spores and products of metabolism from both are easily entrained into the air stream.

As matter encrusts a heat exchanger, its heat exchange efficiency is compromised. The efficiency reduction does not result in an energy reduction. Instead, in the case of a cooling coil, the cooling coil must be made to be cooler or run longer, both of which require more energy for the same unit of work. Likewise, in the case of a heating coil, the heating coil must be made to be hotter or run longer, both of which require more energy for the same unit of work. Moreover, more energy is required to push air across the encrusted heat exchanger, resulting in an increased pressure drop, thus further reducing efficiency. Therefore, either the fan speed must be increased, the motor horsepower increased, or both, or an oversized fan and motor be installed to achieve desired temperature controlled results.

Conventional methods of controlling the accumulation and growth of substrate and microorganisms includes using high-pressure sprayers, surfactants, acids and biocidal agents by applying them to all growth surfaces of an HVAC system. However, the surfactants, acids and biocidal agents are dangerous chemicals and the distribution and use of biocidal agents and acids are strictly controlled by the Environmental Protection Agency (EPA). Thus, those who supply and apply these materials must use masks, gloves and gowns when handling them. These chemicals are dangerous enough that the HVAC system must be shut down and the building vacated during application. Conventional treatment can be extremely expensive.

Recently, a trend has developed using ultraviolet (UV) light sources such as UV emitting low pressure lamps for disinfection/cleaning of water, air and/or cooling coils. However, current systems use light sources having a predominately linear shape. Linear lights are used in air conditioning systems to effectuate irradiation of the cooling coils to avoid growth of mold on the coil that will typically degrade the heat transfer capability of the coil. Moreover, it can lead to an increase in allergic environments in a given dwelling or building associated with a degraded HVAC system.

Tube Luminescence (TL) lamps are linear and typical of current industry practice. In a double ended TL lamp, problems arise in installation and replacement. Many times double end construction lamps are modified into a single ended construction lamp to provide convenience of replacement. Single end lamps can be exchanged from one side rather than two. However, modifying double ended lamps add significant cost to the system. Bi-tube lamps, such as Philips PLL lamps are a major improvement since the electrical contacts terminate at a single end. However, in high air speed environments, bi-tube as well as double ended and single ended TL lamps require some sort of mechanical support. They also add significant resistance to the air stream thereby reducing efficiency.

Generally, air duct systems define relatively small and narrow spaces. Low pressure lamps are relatively long with respect to the narrow space available in a typical air duct system. Installation and replacement of most relatively long low pressure lamps is burdensome and difficult without breaking the lamp. The narrow spaces of the air duct systems make handling the lamps difficult, particularly in the regions where cooling coils are mounted.

Accordingly, a need exists for effective UV light disinfection systems and methods. These and other needs are addressed and/or overcome by the systems and methods of the present disclosure.

The present disclosure provides systems and methods for disinfecting an HVAC system including: (a) an air handling system defining an enclosed air flow path; (b) at least one air moving apparatus positioned within the air duct system for delivering air throughout the air duct system; and (c) at least one non-linear UV light source positioned within the air handling system for disinfecting air passing through the air handling system. The non-linear UV light source is operable to radiate UV light. In an exemplary embodiment, a system according to the present disclosure includes a plurality of non-linear UV light sources positioned within the air handling system. Typically, the air handling system is an air duct system. In an exemplary embodiment, the air handling system includes a temperature coil apparatus positioned within the air handling system and within proximity to the air moving apparatus for controlling temperature of the air delivered throughout the air handling system by the air moving apparatus.

In an exemplary embodiment, at least one non-linear UV light source is positioned within proximity of a temperature coil apparatus positioned within the air handling system for delivering UV light to at least a portion of the surface of the temperature coil apparatus. An exemplary system includes a plurality of non-linear UV light sources wherein at least one of the plurality of non-linear light sources is positioned about the air moving apparatus for delivering UV light to at least a portion of the surface of the air moving apparatus. In an exemplary air handling system at least one of the plurality of non-linear light sources is positioned within the air duct system for radiating UV light as air flow passes across the non-linear light. In an exemplary embodiment, at least one of a plurality of non-linear UV light sources is positioned within proximity of a temperature coil apparatus and an air moving apparatus for radiating UV light to contact any surface surrounding the UV light source including the temperature coil apparatus and the air moving apparatus.

In an exemplary air handling system, a plurality of non-linear UV light sources are positioned throughout the air handling system such that: (i) at least a first non-linear UV light source is positioned within proximity of the temperature coil apparatus; (ii) at least a second non-linear UV light source is positioned about the air moving apparatus; and (iii) at least a third non-linear UV light source is positioned with respect to the surface of the air handling system. The first, second and third non-linear UV light sources are adapted to radiate UV light to contact at least a portion of the surfaces of the temperature coil apparatus, the air moving apparatus and the air handling system respectively.

Examples of non-linear light sources include but are not limited to spherical bulb lamps and circular lamps. A typical air duct system includes four walls assembled to define a substantially rectangular enclosed air flow path. Generally, each of the four walls host at least one non-linear UV light source. An air moving apparatus is typically a fan. In an exemplary embodiment, the non-linear UV light sources are positioned on the four walls such that the fan is substantially surrounded by the non-linear UV light sources to radiate UV light to contact at least a portion of the surface of the fan.

In an exemplary embodiment, the air duct system defines a substantially cylindrical geometry having an inner radius such that a perpendicular cross section of the air flow path defines a substantially circular geometry. In an exemplary cylindrical air duct system, the non-linear UV light source is a circular lamp having an outer radius just less then the inner radius of the cylindrical air duct system so as to reduce air flow disruption created by the presence of the circular lamp.

In an exemplary embodiment, the air moving apparatus is a fan defining an outer diameter and the non-linear UV source is a circular lamp having an inner diameter positioned about the fan such that the inner diameter of the circular lamp substantially surrounds the outer diameter of the fan.

Typically, the coil apparatus can be a plurality of cooling coils or a plurality of heating coils. The UV light source should be operable to disinfect at least a portion of the surface area of the coil apparatus.

In an exemplary embodiment, a plurality of non-linear UV light sources are positioned throughout the air handling system and are in electrical communication with a control unit operable to activate and deactivate the plurality of non-linear light units. Each of the non-linear UV light sources can be coupled to a reflection member positioned adjacent to the non-linear UV light source adapted to intensify and/or direct UV light towards a desired location. An exemplary air handling system according to the present disclosure includes a plurality of reflection members, wherein each of the plurality of non-linear UV light sources is coupled to at least one of the plurality of reflection member positioned adjacent to the non-linear UV light sources adapted to intensify or direct UV light towards a desired location. In an exemplary embodiment, an air handling system according to the present disclosure further includes a dust filter positioned within the air handling system and a non-linear UV light source is positioned within proximity of the dust filter so as to radiate UV light to disinfect the dust filter.

The present disclosure relates to a method of disinfecting components of an HVAC system comprising the steps of: (a) positioning at least one of a plurality of non-linear UV light sources within an air handling system that includes: (i) a temperature coil apparatus adapted to heat and/or cool air passing through the air handling system; and (ii) an air moving apparatus adapted to deliver air throughout the air handling system; and (b) activating the non-linear UV light sources to radiate UV light for disinfecting the air passing through the air handling system and/or disinfecting at least a portion of the surface of the air handling system, the air moving apparatus and/or the temperature coil apparatus.

Additional features, functions and benefits of the disclosed systems and methods will be apparent from the description which follows, particularly when read in conjunction with the appended figures.

To assist those of ordinary skill in the art in making and using the disclosed systems and methods, reference is made to the appended figures, wherein:

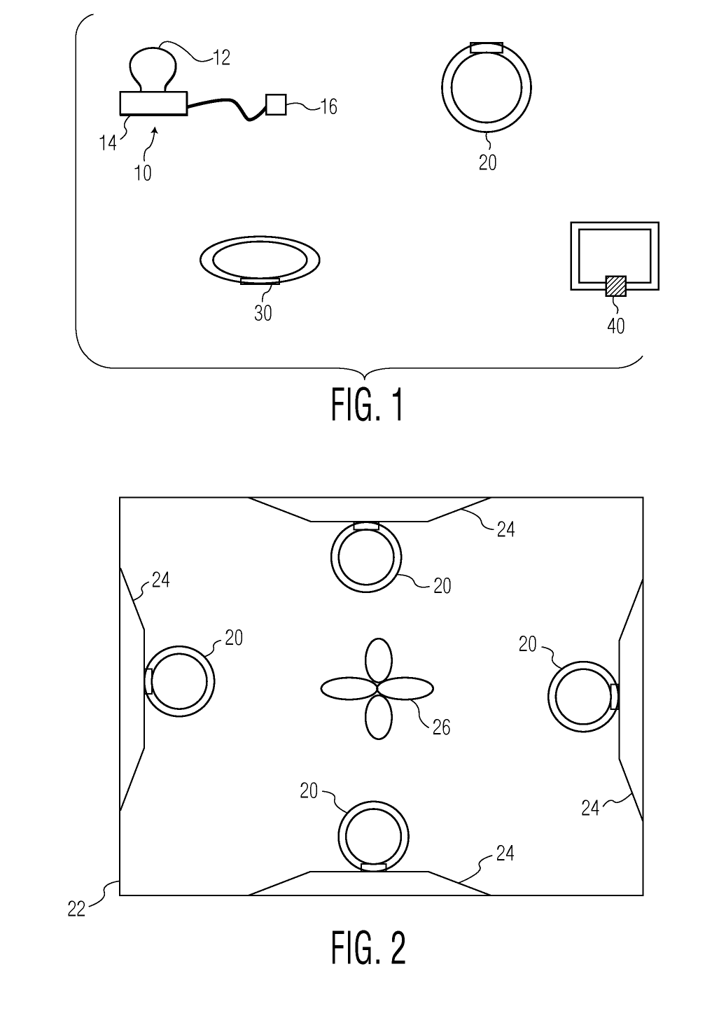

FIG. 1 illustrates exemplary non-linear UV light sources;

FIG. 2 is a schematic illustrating an air duct with a fan surrounded by a plurality of circular UV lamps;

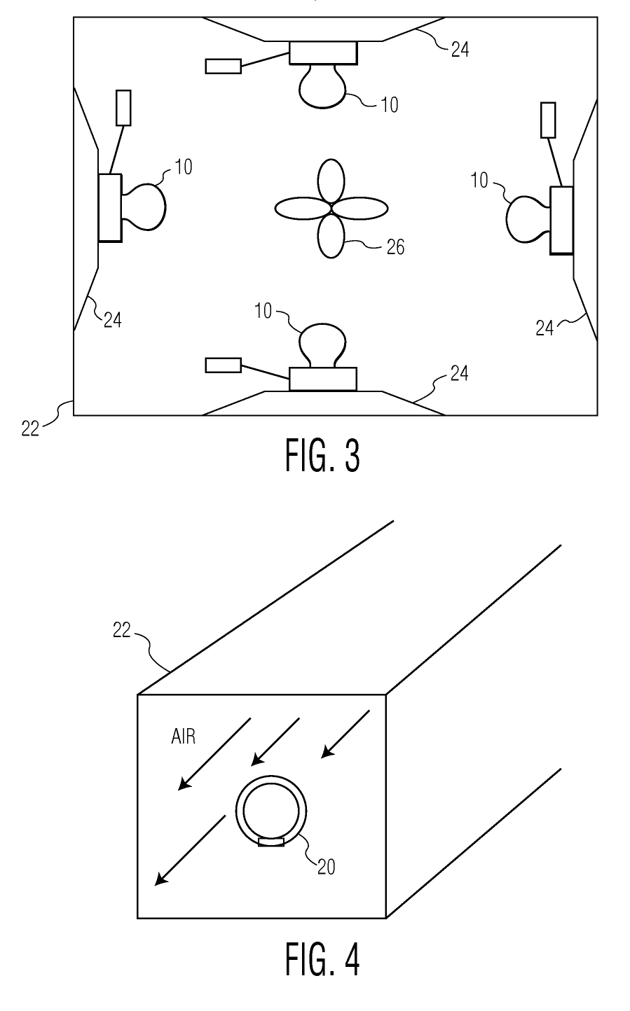

FIG. 3 is a schematic illustrating an air duct with a fan surrounded by a plurality of spherical bulb UV lamps;

FIG. 4 is a perspective view of an exemplary rectangular air duct having a circular UV lamp positioned within the air flow path;

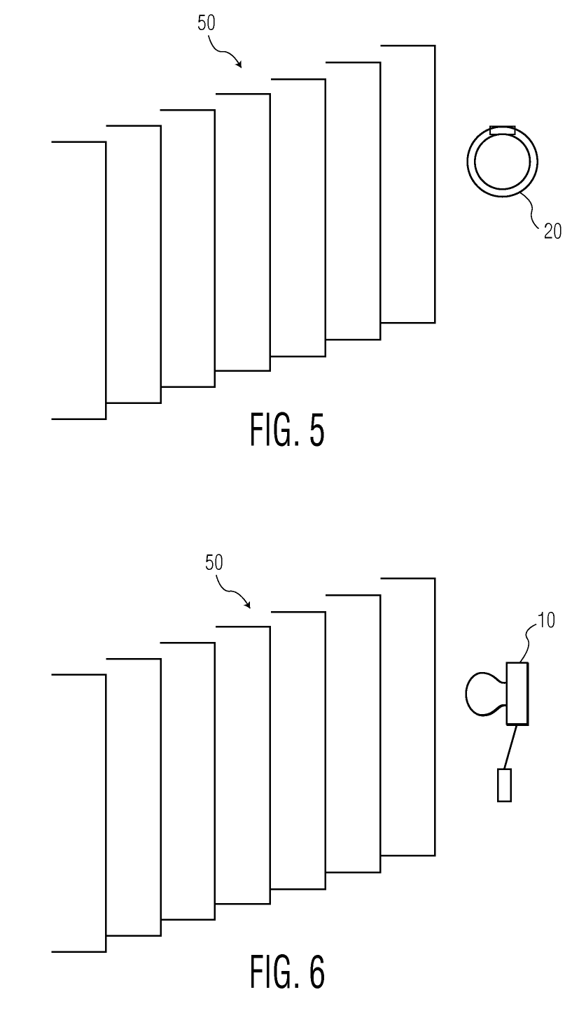

FIG. 5 is a schematic illustrating a circular UV lamp positioned about a temperature coil apparatus;

FIG. 6 is a schematic illustrating a spherical bulb UV lamp positioned about a temperature coil apparatus;

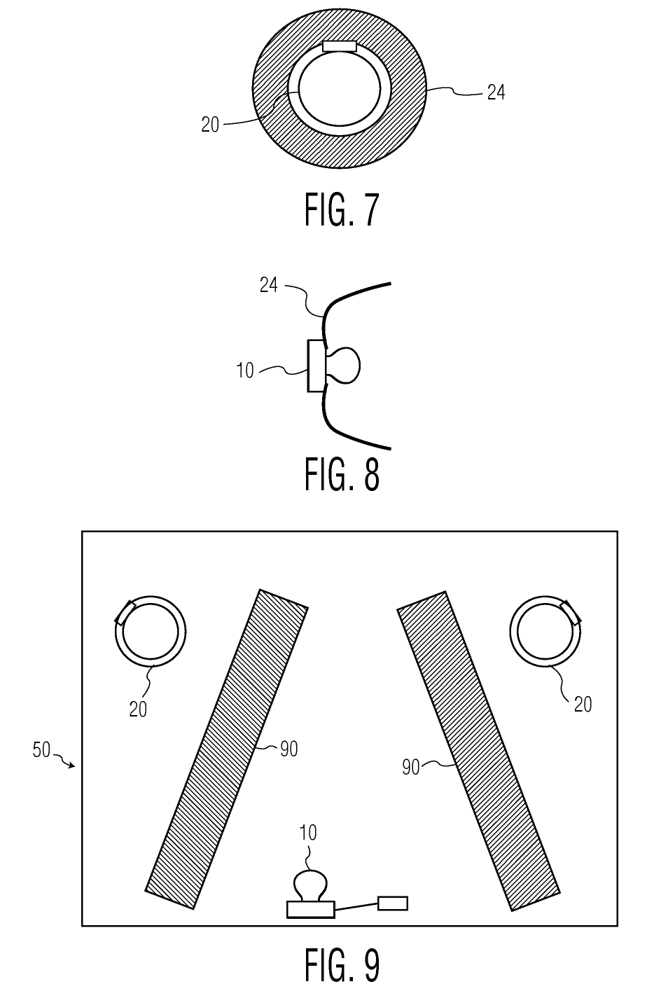

FIG. 7 is a schematic illustrating a circular UV lamp coupled to a reflection member;

FIG. 8 is a schematic illustrating a spherical bulb UV lamp coupled to a reflection member;

FIG. 9 is a schematic illustrating strategic positioning of non-linear UV lamps to disinfect specific locations on a coil apparatus.

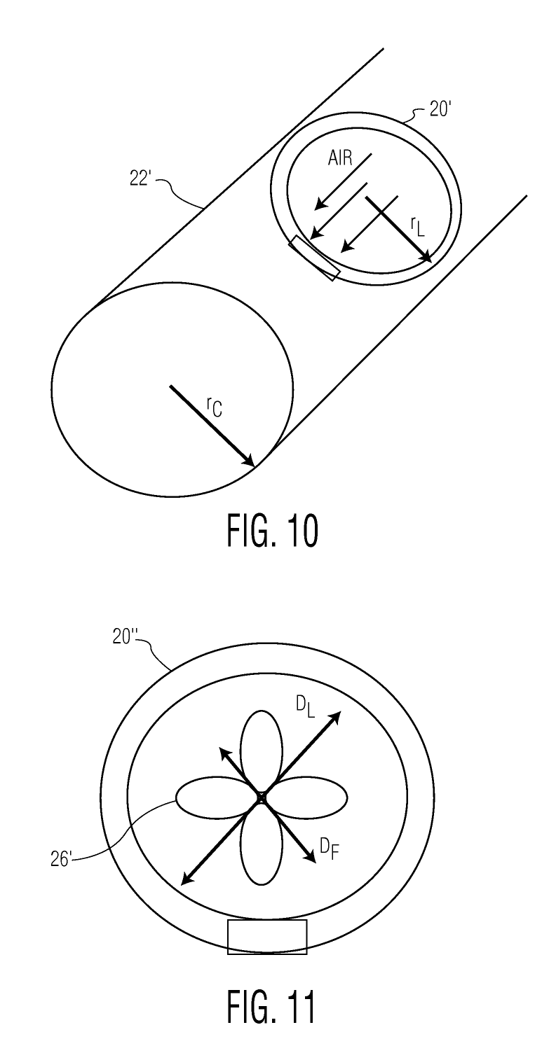

FIG. 10 is a perspective view of an exemplary cylindrical air duct having a circular UV lamp positioned within the air flow path;

FIG. 11 is a schematic illustrating an exemplary circular lamp surrounding a fan.

The present disclosure relates to systems and methods for disinfecting and/or sanitizing different areas associated with HVAC systems using non-linear UV light sources. As discussed herein above, a typical HVAC system includes an air handling system such as air ducts, air delivery apparatuses such as fans and heating and/or cooling cools. In an exemplary embodiment associated with the present disclosure, at least one non-linear UV light source is positioned within an HVAC system such that when the light source is active, UV light is delivered to desired areas of the HVAC system to prevent germ and/or microbial growth.

The light source is active when it is “turned on”. Typically the light source is electrically connected to a power source. In an exemplary embodiment, the light source can be turned on manually by a standard light switch and/or connected to a control system and programmed to turn on according to a schedule. For example, in a particular environment, the lights are programmed to activate once a day for an extended period of time.

Parameters such as activation frequency and activation time can be pre-set by a system user and/or programmer. Factors that may effect these parameters include but are not limited to environmental conditions such as: (i) ambient temperature; (ii) outdoor temperature; (iii) humidity; (iv) typical biological growth for a given area such as spore, pollen or mold growth; and (v) air quality. Additional factors that may effect parameters such as frequency and activation include but are not limited to HVAC system conditions such as: (i) size of a systems duct work; (ii) size of a systems fan or fans; (iii) quantity of fans; (iv) size and configuration of a systems heating and/or cooling coils; and (v) number of heating and/or cooling sections in the system.

An HVAC disinfection system according to the present disclosure includes using non-linear UV light sources for cleaning of cooling coils. Non-linear light sources can be placed in several areas of a typical HVAC system. Examples of effective ways of cleaning areas of an exemplary HVAC system include but are not limited to: positioning at least one non-linear light source in effective proximity of at least one air moving system such as a fan associated with the HVAC system; (ii) positioning at least one non-linear light source in effective proximity of cooling and/or heating coils associated with the HVAC system; and/or (iii) positioning at least one non-linear light source in an effective location within the duct system such that an effective amount of air flow passes over the light source to be disinfected.

FIG. 1 illustrates exemplary schematic geometries of non-linear UV light sources associated with the present disclosure. Non-linear UV light sources include but are not limited to: spherical bulb lamps 10; circular lamps 20, elliptical lamps 30; square lamps 40; and point source lamps (not shown) such as LED's.

Bulb lamps can be electrodeless discharge lamps. Bulb lamp 10 can be any spherical bulb lamp operable to transmit UV light when activated. Examples of lamp 10 include a Philips QL TUV disinfection lamp. QL lamp systems 10 uses light generation that combines the basic principals of induction and gas discharge in an A-lamp design. Void of electrodes, exemplary QL lamps 10 can deliver many hours of high quality light. A typical QL system 10 includes three components: (i) a discharge vessel known as the lamp 12; (ii), a power coupler 14; and (iii) a generator 16. Power coupler 14 typically includes a construction base with an antenna (not shown) upon which discharge vessel 12 is mounted onto a mounting flange (not shown). Power coupler 14 is connected to generator 16 through an electrical connection cable. Generator 16 is typically defined as an electronics inclusive housing. Typically QL systems 10 require an appropriate fixture (not shown). QL systems 10 can typically operate over a wide range of temperatures.

Circular lamp 20 can be an UV TL circular lamp such as a Philips TLE TUV lamp. FIG. 2 is a schematic of an air duct 22 having four interior walls enclosing an air flow path of an exemplary HVAC system. A fan 26 is positioned in the center of air duct 22 adapted to deliver air throughout the HVAC system. In an exemplary embodiment, a plurality of circular lamps 20 are positioned in effective proximity of fan 26. When lamps 20 are activated, UV light is delivered to the surface of fan 26 thereby preventing mold/germ growth. In an exemplary embodiment, four lamps 20 are positioned on each wall of an air duct 22 virtually surrounding fan 26. Exemplary air duct system geometries include but are not limited to circular and/or rectangular.

An exemplary system according to the present disclosure includes at least one reflecting member 24 adapted to direct UV light in a desired direction. Typically, reflecting member 24 is positioned adjacent to lamp 20 such that an effective level of UV transmission is delivered to fan 26. FIG. 2 illustrates an exemplary system having a reflecting member 24 associated with each lamp 20.

FIG. 3 illustrates air duct 22 having four walls enclosing an air flow path. Similar to FIG. 2, a fan 26 is positioned in the center of air duct 22 for delivering air when activated throughout an HVAC system. In an exemplary embodiment, a plurality of spherical bulb UV lamps 10 are positioned in effective proximity of fan 26. When lamps 10 are activated, UV light is delivered to the surface of fan 26 thereby preventing mold/germ growth. In an exemplary embodiment, four lamps 10 are positioned on each wall of an air duct 22 virtually surrounding fan 26.

An exemplary system according to the present disclosure includes at least one reflecting member 24 adapted to direct UV light in a desired direction. Typically, reflecting member 24 is positioned adjacent to lamp 10 such that an effective level of UV transmission is delivered to fan 26. FIG. 3 illustrates an exemplary system having a reflecting member 24 associated with each lamp 10.

FIG. 4 illustrates a perspective view of an exemplary air duct 22. An air quality control system for an exemplary HVAC system according to the present disclosure includes a circular lamp 20 positioned within an air duct 22. Air duct 22 as shown in FIG. 4 is substantially rectangular having four walls defining an air flow path. When lamp 20 is activated, UV light contacts air flow through duct 22 thereby disinfecting the air and improving air quality. The activation of lamp 20 can be programmed to effectuate a desired level of air purity throughout the associated HVAC system. In an exemplary embodiment, a spherical bulb lamp is positioned within the flow path defined by duct 22. A system according to the present disclosure includes a plurality of non-linear lamps positioned at desired locations throughout an HVAC system. For example, circular lamps 20 are positioned in certain air duct locations and spherical bulb lamps are positioned in other locations. In an exemplary system, circular lamps and spherical bulb lamps are positioned through out an air duct system in an alternating fashion.

Non-linear light sources should be securely mounted within the air duct system such that they will withstand heavy air flow. Using circular lamps and/or spherical bulb lamps significantly reduces flow disruption with respect to linear light sources. When air flow is disrupted, the HVAC system operates inefficiently. Non-linear light sources typically occupy less volume and thus provide for less air flow disruption. Moreover, both circular lamps and spherical bulb lamps are single end electrical contact constructions. Single end constructions make installation and bulb/apparatus exchange and or replacement much less difficult than double ended constructions. Single end constructions of non-linear light sources require less mechanical stabilization thus reducing the amount of hardware needed to secure the lamp within the duct.

FIG. 5 and 6 illustrate a schematic of an exemplary heating/cooling coil apparatus 50 associated with the present disclosure. Heating/cooling coil apparatus 50 is typically positioned with an air handling system of an HVAC system. Coil apparatus 50 can be found in a typical HVAC system and is generally a main source for mold/germ growth as discussed herein above. FIG. 5 shows an exemplary circular UV lamp 20 positioned in proximity to coil apparatus 50. FIG. 6 shows an exemplary spherical bulb lamp 10 positioned in proximity to coil apparatus 50. In an exemplary embodiment, a plurality of non-linear light sources are strategically positioned about coil apparatus 50 to effectively disinfect the coils as shown in FIG. 9.

FIG. 7 and 8 illustrate a reflection member 24 positioned adjacent to non-linear lamps. In FIG. 7, reflection member 24 defines a substantially circular geometry to provide appropriate light guidance to circular lamp 20. In FIG. 8, member 24 defines a curved geometry to provide appropriate light guidance to spherical bulb lamp 10. In an exemplary embodiment, reflection members 24 can be included in FIG. 5 and 6 to reflect light transmitted from the non-linear light source and direct the transmitted light towards coil apparatus 50. Reflection members 50 should be designed to improve light transmission efficiency to ensure that most of the light transmitted from the non-linear light source is directed to desired locations.

FIG. 9 illustrates an exemplary schematic of positioning a plurality of non-linear light sources in strategic locations about a coil apparatus 50. Coil apparatus 50 includes a plurality of coils 90. Positioned about coils 90 are circular lamps 20 and a spherical bulb lamp 10. Strategically positioning non-linear lamps about coils 90 allows for direct UV light manipulation to disinfect hard to reach areas typical of unwanted growth. Since non-linear light sources include a variety of geometries, a system according to the present disclosure can disinfect substantially more surface area of a coil apparatus than linear light sources. Thus, an HVAC system having a plurality of non-linear light sources strategically positioned about a coil apparatus will substantially improve the coils life and efficiency.

FIG. 10 illustrates a perspective view of an exemplary air duct 22′. An air quality control system for an exemplary HVAC system according to the present disclosure includes a circular lamp 20′ positioned within an air duct 22′. Air duct 22′ as shown in FIG. 4 is substantially cylindrical such that a perpendicular cross section with respect to the air flow path defines a substantially circular geometry. When lamp 20′ is activated, UV light radiates throughout the surrounding area contacting air flow through duct 22′ and at least a portion of the inner surface of air duct 22′ thereby disinfecting the air and improving air quality and disinfecting at least a portion of the inner surface of the air duct 22′. The activation of lamp 20′ can be programmed to effectuate a desired level of air purity throughout the associated HVAC system.

In an exemplary embodiment, a spherical bulb lamp is positioned within the flow path defined by duct 22′. A system according to the present disclosure includes a plurality of non-linear lamps positioned at desired locations throughout an HVAC system. For example, circular lamps 20′ are positioned in certain air duct locations and spherical bulb lamps are positioned in other locations. In an exemplary system, circular lamps and spherical bulb lamps are positioned through out an air duct system in an alternating fashion.

FIG. 10 shows an exemplary air duct 22′ having an inner radius R_{C}. Circular lamp 20′ is shown having an outer radius R_{L}. In an exemplary embodiment, R_{L} and R_{C} are almost the same size such that R_{L} is just less then R_{C}. Positioning a circular lamp 20′ dimensioned to almost have the same outer radius as the inner radius of the air duct 22′ advantageously reduces the amount of air resistance created by the placement of a circular lamp 20′ within the air flow path. In an exemplary embodiment, R_{L} is substantially equal to R_{C} such that the outer surface of lamp 20′ is flush with the inner surface of duct 22′. Positioning lamp 20′ flush with air duct 22′ allows for additional heat transfer from the lamp 20′ to the surface of the duct 22′. Adding additional heat transfer allows for improving the efficiency and life of lamp 20′.

FIG. 11 illustrates a schematic of an exemplary positioning of a circular lamp 20″ having an inner diameter D_{L} surrounding a fan 26′ having an outer diameter D_{F}. An exemplary fan 26′ can be positioned within the inner circle of an exemplary lamp 20″ such that an D_{L} is greater than D_{F} and the UV light radiating from lamp 20″ delivers UV light to the surface of fan 26′ and surrounding air duct surfaces.

In an exemplary embodiment, one non-linear UV light source is positioned within an exemplary air handling system within relative proximity to at least a portion of the inner surface of the air handling system, an air moving apparatus such as a fan positioned within the air handling system, and a temperature coil apparatus positioned within the air handling system. The non-linear UV light source is adapted to radiate UV light so that at least a portion of the surfaces of each of the components within proximity of the non-linear UV light source are contacted by UV light thereby being disinfected.

An exemplary HVAC system according to the present disclosure includes a dust filter. Non-linear UV light sources may be positioned within proximity of the dust filter thereby disinfecting the dust filter by radiating UV light to contact the surface of the dust filter.

In an exemplary embodiment, the HVAC system includes air ducts made from advantageously reflective materials such as aluminum. The reflective material should be effective in reflecting UV light generated from at least one non-linear UV light source positioned within the air ducts throughout the air ducts. This is especially advantageous in a cylindrical air duct embodiment since rounded surfaces are typically highly reflective. Reflective materials can improve the efficiency of delivering the UV light throughout the air ducts thereby improving the efficiency of the disinfecting function of the non-linear UV light source.

An exemplary HVAC system according to the present disclosure includes air ducts, at least one air moving or delivery apparatus such as a fan and at least one of a heating or cooling coil apparatus. A plurality of non-linear light sources are strategically positioned throughout the HVAC system including: (i) the air ducts to improve air quality; (ii) effective proximity of the at least one air moving or delivery apparatus to disinfect and prevent undesired growth on the surface of the air moving apparatus; and (iii) about the at least one coil apparatus to disinfect and prevent undesired growth on the surface of the coil apparatus.

Non-linear UV light sources such as a spherical bulb or a circular lamp are compact and single end constructions thus providing increased positioning options and better accessibility throughout an HVAC system. Moreover, they are easier to install and replace compared to linear light sources. The compact size of the non-linear light sources also creates much less air resistance compared to linear light sources thereby allowing the HVAC system to operate more efficiently. The QL lamp described above has an exceptionally long life cycle thus, drastically reducing replacement costs. The lamps should be fabricated to function within a given environments temperature range. Non-linear lamps can also be positioned about a dispensing tray associated with a cooling coil apparatus to prevent undesired growth in the tray. An exemplary system according to the present disclosure includes programming a plurality of non-linear lights positioned throughout an HVAC system to shut off or deactivate during detected undesired environmental conditions. Thus, a control system can detect temperatures within the system which may reduce lamp efficiency and accordingly send a signal to the system to deactivate. Systems and/or methods according to the present disclosure can be programmed to coordinate during HVAC system shutdown to prevent operation at non efficient temperature.

Although the present disclosure has been described with reference to exemplary embodiments and implementations thereof, the disclosed systems and methods are not limited to such exemplary embodiments/implementations. Rather, as will be readily apparent to persons skilled in the art from the description provided herein, the disclosed systems and methods are susceptible to modifications, alterations and enhancements without departing from the spirit or scope of the present disclosure. Accordingly, the present disclosure expressly encompasses such modification, alterations and enhancements within the scope hereof.

1. A system for disinfecting components of an HVAC system, comprising:
(a) an air handling system defining an enclosed air flow path;
(b) at least one air moving apparatus positioned within the air duct system for delivering air throughout the air duct system; and
(c) at least one non-linear UV light source positioned within the air handling system for disinfecting air passing through the air handling system;
wherein the non-linear UV light source is operable to radiate UV light.

2. A system according to claim 1, further comprising a plurality of non-linear UV light sources positioned within the air handling system.

3. A system according to claim 2, wherein the air handling system is an air duct system.

4. A system according to claim 2, further comprising at least one temperature coil apparatus positioned within the air handling system and within proximity to the air moving apparatus for controlling temperature of the air delivered throughout the air handling system by the air moving apparatus.

5. A system according to claim 4, wherein at least one of the plurality of non-linear UV light sources is positioned within proximity of the temperature coil apparatus for delivering UV light to at least a portion of the surface of the temperature coil apparatus.

6. A system according to claim 2, wherein at least one of the plurality of non-linear light sources is positioned about the air moving apparatus for delivering UV light to at least a portion of the surface of the air moving apparatus.

7. A system according to claim 4, wherein at least one of the plurality of non-linear UV light sources is positioned within proximity of the temperature coil apparatus and the air moving apparatus for radiating UV light to contact any surface surrounding the UV light source including the temperature coil apparatus and the air moving apparatus.

8. A system according to claim 4, wherein the plurality of non-linear UV light sources are positioned throughout the air handling system such that:
(i) at least a first non-linear UV light source is positioned within proximity of the temperature coil apparatus;
(ii) at least a second non-linear UV light source is positioned about the air moving apparatus; and
(iii) at least a third non-linear UV light source is positioned with respect to the surface of the air handling system; and
wherein the first, second and third non-linear UV light sources are adapted to radiate UV light to contact at least a portion of the surfaces of the temperature coil apparatus, the air moving apparatus and the air handling system respectively.

9. A system according to claim 1, wherein the non-linear light source is a spherical bulb lamp.

10. A system according to claim 1, wherein the non-linear light source is a circular lamp.

11. A system according to claim 3, wherein the air duct system includes four walls assembled to define a substantially rectangular enclosed air flow path.

12. A system according to claim 11, wherein each of the four walls host at least one non-linear UV light source.

13. A system according to claim 12, wherein the air moving apparatus is a fan.

14. A system according to claim 13, wherein the non-linear UV light sources are positioned on the four walls such that the fan is substantially surrounded by the non-linear UV light sources to radiate UV light to contact at least a portion of the surface of the fan.

15. A system according to claim 3, wherein the air duct system defines a substantially cylindrical geometry having an inner radius such that a perpendicular cross section of the air flow path defines a substantially circular geometry.

16. A system according to claim 15, wherein the non-linear UV light source is a circular lamp having an outer radius just less then the inner radius of the cylindrical air duct system so as to reduce air flow disruption created by the presence of the circular lamp.

17. A system according to claim 1, wherein the air moving apparatus is a fan defining an outer diameter and the non-linear UV source is a circular lamp having an inner diameter positioned about the fan such that the inner diameter of the circular lamp substantially surrounds the outer diameter of the fan.

18. A system according to claim 4, wherein the coil apparatus is a plurality of cooling coils.

19. A system according to claim 4, wherein the coil apparatus is a plurality of heating coils.

20. A system according to claim 8, wherein the plurality of non-linear light sources positioned throughout the air handling system are in electrical communication with a control unit operable to activate and deactivate the plurality of non-linear light units.

21. A system according to claim 1, wherein the non-linear UV light source is coupled to an adjacent reflection member adapted to intensify and/or direct UV light towards a desired location.

22. A system according to claim 8, further comprising a plurality of reflection members, wherein each of the plurality of non-linear light sources is coupled to at least one of the plurality of reflection members positioned adjacent to the non-linear UV light sources adapted to intensify and/or direct UV light towards a desired location.

23. A system according to claim 1, further including a dust filter positioned within the air handling system, wherein the non-linear UV light source is positioned within proximity of the dust filter so as to radiate UV light to disinfect the dust filter.

24. A system according to claim 3, wherein the air duct system is fabricated from a reflective material.

25. A system according to claim 23, wherein the reflective material is aluminum.

26. A method of disinfecting components of an HVAC system comprising the steps of:
(a) positioning at least one of a plurality of non-linear UV light sources within an air handling system, the air handling system including: (i) a temperature coil apparatus adapted to heat and/or cool air passing through the air handling system; and (ii) an air moving apparatus adapted to deliver air throughout the air handling system;
(b) activating the non-linear UV light sources to radiate UV light for disinfecting the air passing through the air handling system and/or disinfecting at least a portion of the surface of the air handling system, the air moving apparatus and/or the temperature coil apparatus.
